SEQUENCE LISTING (6 sequences)

SEQ ID NO: 1 - protein, 31 aa, unidentified
  REGION 1..31: note = source = /note="Description of Unknown: GLP-1 polypeptide"
HAEGTFTSDVSSYLEGQAAKEFIAWLVKGRG

SEQ ID NO: 2 - protein, 39 aa, unidentified
  REGION 1..39: note = source = /note="Description of Unknown: Exendin-3 polypeptide"
HSDGTFTSDLSKQMEEEAVRLFIEWLKNGGPSSGAPPPS

SEQ ID NO: 3 - protein, 39 aa, unidentified
  REGION 1..39: note = source = /note="Description of Unknown: Exendin-4 polypeptide"
HGEGTFTSDLSKQMEEEAVRLFIEWLKNGGPSSGAPPPS

SEQ ID NO: 4 - protein, 30 aa, unidentified
  REGION 1..30: note = source = /note="Description of Unknown: Exendin-4 polypeptide"
HGEGTFTSDLSKQMEEEAVRLFIEWLKNGG

SEQ ID NO: 5 - protein, 39 aa, unidentified
  REGION 1..39: note = source = /note="Description of Unknown: Exendin-4 polypeptide"
HGEGTFTSDLSKQLEEEAVRLFIEFLKNGGPSSGAPPPS

SEQ ID NO: 6 - protein, 28 aa, unidentified
  REGION 1..28: note = source = /note="Description of Unknown: Exendin-4 peptide"
HGEGTFTSDLSKQLEEEAVRLAIEFLKN

(19) United States
(12) Patent Application Publication
(10) Pub. No.: US 2023/0390195 A1
(43) Pub. Date: Dec. 7, 2023

(54) STABLE COMPOSITIONS FOR INCRETIN MIMETIC COMPOUNDS

(71) Applicant: Delpor, Inc., Brisbane, CA (US)

(72) Inventor: Gregory A. Watkins, Oakland, CA (US)

(21) Appl. No.: 18/328,678

(22) Filed: Jun. 2, 2023

Related U.S. Application Data
(63) Continuation of application No. 18/149,592, filed on Jan. 3, 2023.
Application No. 18/149,592 is a continuation of application No. 16/336,086, filed on Mar. 22, 2019, now abandoned.
(60) Provisional application No. 62/398,964, filed on Sep. 23, 2016.

(51) Int. Cl. A61K 9/10; A61K 9/00; A61K 47/12; A61K 38/26; A61P 3/10

(52) U.S. Cl. CPC A61K 9/10; A61K 9/0024; A61K 47/12; A61K 38/26; A61K 9/0019; A61P 3/10

(57) ABSTRACT

A composition comprising an aqueous suspension comprising an incretin mimetic and an organic acid is described. The organic acid is one that (i) has a water solubility at room temperature of between about 0.01 and 10 g/L, (ii) has a molar mass of less than about 500 grams per mole, and/or (iii) maintains a pH of the suspension in its environment of use of between 3.0-6.0 for a period of at least about 30 days, and stabilizes the incretin mimetic to provide a composition that is suitable for delivering the compound in a biologically active or potent form for a sustained period of time. Devices comprising the compositions and methods of treatment are also described.

### CROSS-REFERENCE TO RELATED APPLICATIONS

This application is a continuation of U.S. patent application Ser. No. 18/149,592 filed Jan. 3, 2023, currently pending, which is a continuation of U.S. patent application Ser. No. 16/336,086, filed Mar. 22, 2019, now abandoned, which claims the benefit of U.S. National Stage Application under U.S.C. § 371 to International Patent Application No. PCT/US2017/053085, filed Sep. 22, 2017, now expired, which claims the benefit of priority to U.S. Provisional Application No. 62/398,964, filed Sep. 23, 2016, now expired, each of which is hereby incorporated by reference in its entirety.

### REFERENCE TO SEQUENCE LISTING, TABLE OR COMPUTER PROGRAM

The instant application contains a Sequence Listing which has been filed electronically in XML format and is hereby incorporated by reference in its entirety. Said XML copy, created on Jun. 2, 2023, is named 091505-0103.xml and is 8,952 bytes in size.

### TECHNICAL FIELD

The subject matter described herein relates to compositions and formulations for an incretin mimetic, and to drug delivery devices comprising the compositions and formulations for controlled, sustained delivery of the incretin mimetic.

### BACKGROUND

Peptides and proteins degrade via a number of different mechanisms, including deamidation, oxidation, hydrolysis, disulfide exchange and racemization. These degradation mechanisms must be considered when designing a formulation to deliver a protein or peptide for a sustained period of time. Further complicating formulation design of peptides and proteins is that aqueous solvents can drive aggregation and precipitation processes, particularly at high solute concentrations. In some cases, these peptide/protein aggregates may be immunogenic or toxic; in others, they may sequester the therapeutic agent in a kinetically stable, inactive form. The high polarity of water and its ability to function as a weak nucleophile can also accelerate numerous chemical decomposition processes that take place through polar intermediates, including the hydrolysis of peptide bonds. Therefore, it is a challenge to provide a formulation comprising a peptide or a protein that is both physically and chemically stable over time at ambient or physiological temperatures. One approach is to provide a dried formulation, where the peptide or protein is spray-dried or lyophilized. Dry powder peptide formulations often exhibit stable biological activity over time relative to aqueous formulations at ambient and/or at physiological temperatures. However, dry peptide or protein formulations are often unsuitable when the formulation is to be delivered via an implantable drug delivery device, especially where the peptide or protein is released from the device by a diffusion controlled-released mechanism, as the peptide or protein needs to be in solution in order to diffuse from the device. Thus, there remains a need for liquid formulations in which a therapeutically active peptide or protein is stable (e.g., with a retention of potency ≥about 70%) over a period of several weeks (e.g., 2 weeks or 3 weeks) to at least about two months.

### BRIEF SUMMARY

The following aspects and embodiments thereof described and illustrated below are meant to be exemplary and illustrative, not limiting in scope.

In one aspect, a composition, comprising an aqueous suspension or slurry, is provided. The aqueous suspension or slurry comprises an incretin mimetic and an organic acid that (i) has a water solubility at room temperature of less than about 20 g/L and (ii) maintains a pH of the composition in its environment of use of between 3.0-6.0 for a period of at least about 30 days.

In one aspect, a composition, comprising an aqueous suspension or slurry, is provided. The aqueous suspension or slurry comprises an incretin mimetic and one or more organic acids that (i) has a water solubility at room temperature between about 0.01 and 10 g/L or less than about 20 g/L, (ii) a molar mass of less than 500 grams per mole, and (iii) maintains a pH of the composition in its environment of use of between 3.0-6.0 for a period of at least about 30 days.

In one embodiment, the aqueous suspension is a heterogeneous mixture comprising the incretin mimetic and the organic acid, where the organic acid maintains the pH of the aqueous fraction of the mixture in its environment of use for the stated period. In one embodiment, the environment of use is in vivo. In another embodiment, the environment of use is in vitro in a release medium maintained at 37° C.

In one embodiment, the organic acid is present in an amount above its saturation concentration at the end of the period.

In another embodiment, the organic acid is crystalline and has a melting temperature of more than about 37° C.

In yet another embodiment, the incretin mimetic is a glucagon-like peptide-1 (GLP-1) agonist.

In other embodiments, the GLP-1 agonist is exendin or an exendin-analogue. An exemplary GLP-1 agonist is exendin-4.

In still other embodiments, the incretin mimetic at the beginning of the period is at a concentration in the solution (or suspension, or slurry) of greater than or equal to 1 mg/mL.

In yet another embodiment, the incretin mimetic at the beginning of the period is at a concentration in the solution (or suspension, or slurry) of greater than or equal to 10 mg/mL.

In one embodiment, the aqueous suspension or slurry comprises or is prepared from (or manufactured with) an organic acid being suspended in a water-based solution, such as an aqueous buffered solution. An example is phosphate buffered saline.

In one embodiment, the organic acid is an aromatic carboxylic acid. Exemplary acids, in one embodiment, are those having a carboxylic acid group bound to an unsubstituted benzene or pyridine ring. In one embodiment, the carboxylic acid is selected from the group consisting of benzoic acid, picolinic acid, nicotinic acid, and isonicotinic acid.

In another embodiment, the carboxylic acid is one having a benzene ring and one electron-donating group. In another embodiment, the carboxylic acid has antioxidant properties.

In still another embodiment, the carboxylic acid is selected from the group consisting of o-anisic acid, m-anisic acid, p-anisic acid, p-aminobenzoic acid (PABA), o-aminobenzoic acid (anthranilic acid), o-toluic acid, m-toluic acid, p-toluic acid and salicylic acid.

In another embodiment, the carboxylic acid is an aromatic carboxylic acid with a benzene ring and two electron donating groups. In another embodiment, the carboxylic acid has antioxidant properties. In one embodiment, and by way of example, the carboxylic acid is vanillic acid.

In yet another embodiment, the carboxylic acid is one having at least two carboxylic acid groups bonded to a benzene ring. In one embodiment, and by way of example, the carboxylic acid is terephthalic acid.

In yet another embodiment, the carboxylic acid is one having a carboxylic acid group bonded to a naphthalene or quinoline ring. In one embodiment, and by way of example, the carboxylic acid is selected from the group consisting of 1-naphthoic acid, 2-naphthoic acid, quinaldic acid, 3-quinolinecarboxylic acid, 4-quinolinecarboxylic acid, 5-quinolinecarboxylic acid, 6-quinolinecarboxylic acid, 7-quinolinecarboxylic acid, and 8-quinolinecarboxylic acid.

In another embodiment, the carboxylic acid is one having an electron-donating group selected from the group consisting of hydroxy, methoxy, amino, alkylamino, dialkylamino, and alkyl. In one embodiment, and by way of example, the carboxylic acid is selected from the group consisting of 6-hydroxy-2-naphthoic acid, 6-hydroxy-3-naphthoic acid, 8-hydroxy-2-quinolinecarboxylic acid and 8-hydroxy-7-quinolinecarboxylic acid.

In yet another embodiment, the carboxylic acid is one having one or two carboxylic acid groups directly bonded to a biphenyl ring system. In one embodiment, and by way of example, the carboxylic acid is selected from the group consisting of 2-phenylbenzoic acid, 3-phenylbenzoic acid, 4-phenylbenzoic acid and diphenic acid.

In yet another embodiment, the carboxylic acid is one having at least one additional electron donating substituent on the biphenyl carboxylic acid moiety. In one embodiment, and by way of example, the carboxylic acid is selected from the group consisting of 4′-hydroxy-4-biphenylcarboxylic acid, 4′-hydroxy-2-biphenylcarboxylic acid, 4′-methyl-4-biphenylcarboxylic acid, 4′-methyl-2-biphenylcarboxylic acid, 4′-methoxy-4-biphenylcarboxylic acid, and 4′-methoxy-2-biphenylcarboxylic acid.

In still another embodiment, the carboxylic acid is one having a carboxylic acid functional group separated from a benzene, pyridine, naphthalene, or quinoline ring by a chain of 1-4 saturated carbon atoms. In one embodiment, and by way of example, the carboxylic acid is phenylacetic acid or 3-phenylpropionic acid.

In another embodiment, the carboxylic acid is an aliphatic dicarboxylic acid with 4-8 carbons positioned between the carboxylic acid groups. In another embodiment, the carboxylic acid is an aliphatic dicarboxylic acid containing 6-10 carbon atoms. In one embodiment, and by way of example, the carboxylic acid is selected from the group consisting of adipic acid ((CH₂)₄COOH)₂), pimelic acid (HO₂C(CH₂)₅CO₂H), suberic acid (HO₂C(CH₂)₆CO₂H), azelaic acid (HO₂C(CH₂)₇CO₂H), and sebacic acid (HO₂C(CH₂)₈CO₂H).

In another embodiment, the carboxylic acid is an unsaturated or polyunsaturated dicarboxylic acid containing 4-10 carbons. In one embodiment, and by way of example, the carboxylic acid is selected from the group consisting of fumaric acid, trans,trans-muconic acid, cis,trans-muconic acid, and cis,cis-muconic acid.

In other embodiments, the carboxylic acid is a cis-cinnamic acid or a trans-cinnamic acid. In still other embodiments, the carboxylic acid is a trans-cinnamic acid with one, two, or three electron-donating groups selected from hydroxy, methoxy, amino, alkylamino, dialkylamino, or alkyl groups. In yet other embodiments, the trans-cinnamic acid is selected from the group consisting of o-coumaric acid, m-coumaric acid, p-coumaric acid, o-methylcinnamic acid, m-methylcinnamic acid, p-methylcinnamic acid, o-methoxycinnamic acid, m-methoxycinnamic acid, and p-methoxycinnamic acid, and ferulic acid.

In one embodiment, the organic acid is a phenol or a naphthol substituted with between about 2-5 electron-withdrawing groups such as —F, —Cl, —Br, —I, —CN, —CHO, and —NO₂. In one embodiment, and by way of example, the organic acid is pentafluorophenol or 2,4-dinitrophenol.

In another embodiment, the organic acid is a 1,3-dicarbonyl compound containing an acidic CH bond (pKa<8). In one embodiment, and by way of example, the organic acid is 2,2-dimethyl-1,3-dioxane-4,6-dione (Meldrum's acid), cyanuric acid, or barbituric acid.

In still another embodiment, the organic acid is an imide. In one embodiment, and by way of example, the imide is phthalimide or a substituted phthalimide. In another embodiment, the substituted phthalimide bears at least one electron-withdrawing substituent.

In yet another embodiment, the organic acid is a hydroxamic acid. In one embodiment, and by way of example, the hydroxamic acid is an aromatic hydroxamic acid containing one hydroxamic functional group bonded directly to an aromatic ring. In one embodiment, the aromatic ring is selected from the group consisting of a benzene ring, a pyridine ring, a naphthalene ring, a quinoline ring, and a biphenyl ring. In still another embodiment, the hydroxamic acid is benzhydroxamic acid. In yet another embodiment, the hydroxamic acid is one containing a hydroxamic functional group separated from an aromatic ring by a chain of 1-4 sp³-hybridized carbon atoms.

In yet another embodiment, the aromatic ring is selected from the group consisting of a benzene ring, a pyridine ring, a naphthalene ring, a quinoline ring, and a biphenyl ring.

In still another embodiment, the hydroxamic acid is a dihydroxamic acid containing two or more hydroxamic acid functional groups bonded directly to a benzene ring, a pyridine ring, a naphthalene ring, a quinoline ring, or a biphenyl ring system.

In other embodiments, the hydroxamic acid contains an aromatic ring substituted with an electron donating substituent selected from hydroxy, methoxy, amino, alkylamino, dialkylamino, and alkyl groups.

In other embodiments, the hydroxamic acid is an aliphatic dihydroxamic acid containing 6-carbon atoms.

The hydroxamic acid is, in one embodiment, suberohydroxamic acid.

The hydroxamic acid is, in other embodiments, an unsaturated dihydroxamic acid containing 6-10 carbon atoms.

In another embodiment, the aromatic carboxylic acid is selected from the group consisting of 3-phenylpropionic acid, cinnamic acid, a hydroxy-derivative of cinnamic acid, a methoxy derivative of cinnamic acid, nicotinic acid, benzoic acid, an amino-derivative of benzoic acid, a methoxy derivative of benzoic acid, and terephthalic acid.

In yet another embodiment, the hydroxy-derivative of cinnamic acid is m-coumaric acid or p-coumaric acid.

In yet other embodiments, the p-coumaric acid is trans-p-coumaric acid.

In other embodiments, the methoxy derivative of cinnamic acid is p-methoxycinnamic acid or m-methoxycinnamic acid.

In still other embodiments, the amino-derivative of benzoic acid is o-amino-benzoic acid (anthranilic acid) or 4-aminobenzoic acid (para-aminobenzoic acid; PABA).

In another embodiment, the methoxy derivative of benzoic acid is 4-methoxybenzoic acid (p-anisic acid), o-anisic acid or m-anisic acid.

In one embodiment, the incretin mimetic in the suspension or slurry maintains at least about 70% of its potency for the period at 37° C.

In one embodiment, the composition is in dry form. In another embodiment, the composition is in dry form and hydrates in situ when in its environment of use.

In another aspect, a device comprising a composition as described herein is provided. The device is configured for subcutaneous implantation into a mammal.

In another aspect, an implantable device is provided. The device comprises a reservoir comprising a formulation of an incretin mimetic, the formulation comprising (i) an amount of the incretin mimetic to provide substantially zero-order release of the incretin mimetic for a delivery period of at least about 30 days and at a rate that provides a therapeutic effect and (ii) an organic acid that (a) maintains a pH of the formulation when hydrated in its environment of use of between 3.0-6.0 for the delivery period and (b) is present at the end of the delivery period in an amount above its saturation concentration in the formulation when hydrated.

In one embodiment, the formulation is in dry form. In various embodiments, and by way of example, the formulation is a powder, a tablet or a film; or a mixture of two or more powders, tablets, or films.

In another embodiment, the formulation hydrates in the presence of an aqueous solution to form an aqueous suspension or slurry. In one embodiment, the aqueous solution is in vivo fluid.

In one embodiment, the formulation when hydrated has the property that less than 30% of the incretin mimetic degrades when stored for 3 months at 37° C.

In another embodiment, the incretin mimetic is released from the device at a rate that provides a therapeutically effect for the period.

In still another embodiment, the organic acid has a water solubility at room temperature of less than about 20 g/L. In another embodiment, the organic acid has a water solubility at room temperature of less than about 10 g/L and a molar mass of less than 500 grams per mole.

In another embodiment, the organic acid has a water solubility at room temperature of less than about 20 g/L or of less than about 10 g/L and a pKa between 3 and 6. In another embodiment, the organic acid has a water solubility at room temperature of less than about 10 g/L and a molar mass of less than 500 grams per mole and a pKa between 3 and 6.

In yet another embodiment, the organic acid has a melting temperature of greater than about 37° C.

In another aspect, a method for sustained, controlled delivery of an incretin mimetic is provided. The method comprises providing a composition or a device as described herein. In some embodiments, the method further comprises administering the device, such as by subcutaneous implantation.

In another aspect, a method for sustained, controlled delivery of a GLP-1 agonist, such as exendin-4 is provided, where the method providing a composition or a device as described herein. In some embodiments, the method further comprises administering the device, such as by subcutaneous implantation.

In another aspect, a method to lower plasma glucose or to treat diabetes mellitus is provided, where the method provides a composition or a device as described herein. In some embodiments, the method further comprises administering the device, such as by subcutaneous implantation.

In one embodiment, the method is for treating type 2 diabetes mellitus.

In another aspect, a method to reduce food intake or to reduce body weight, or a method for chronic weight management is provided, where the method provides a composition or a device as described herein. In some embodiments, the method further comprises administering the device, such as by subcutaneous implantation.

In one embodiment, the incretin mimetic is liraglutide.

In another aspect, a method to reduce gastric motility or delay gastric emptying is provided, where the method provides a composition or a device as described herein. In some embodiments, the method further comprises administering the device, such as by subcutaneous implantation.

In addition to the exemplary aspects and embodiments described above, further aspects and embodiments will become apparent by reference to the drawings and by study of the following descriptions.

Additional embodiments of the present methods, devices and compositions, and the like, will be apparent from the following description, drawings, examples, and claims. As can be appreciated from the foregoing and following description, each and every feature described herein, and each and every combination of two or more of such features, is included within the scope of the present disclosure provided that the features included in such a combination are not mutually inconsistent. In addition, any feature or combination of features may be specifically excluded from any embodiment of the present invention. Additional aspects and advantages of the present invention are set forth in the following description and claims, particularly when considered in conjunction with the accompanying examples and drawings.

### BRIEF DESCRIPTION OF THE DRAWINGS

FIG. 1 shows selected results from a study to assess the stability of exendin-4 as a function of time (in days), where the exendin-4 was present in a control formulation of phosphate buffered saline (˜2 mg/mL; initial pH=7.4; open diamonds) and in formulations comprised of an aqueous suspension of the peptide and an organic acid with water solubility at room temperature of less than 10 g/L: p-coumaric acid (closed squares), m-coumaric acid (x symbols), p-methoxycinnamic acid (closed triangles), trans-cinnamic acid (open triangles), 4-methylcinnamic acid (asterisks), 4-aminobenzoic acid (PABA) (open circles), citric acid (water soluble acid, as a control, closed diamonds), PBS control (open diamonds). The pH values of active formulations ranged from approximately 4 to 5.4. Control formulations were comprised of PBS (organic acid omitted, pH=7.2; open diamonds) or citric acid at ≥20 g/L (pH=2.0; closed diamonds).

FIG. 2 shows selected results from a study to assess the stability of exendin-4 at two concentrations (2.0 mg/mL or 0.4 mg/mL) in a weakly acidic formulation (pH-4.4) prepared with p-coumaric acid or in a control formulation lacking an organic acid (phosphate buffered saline (pH-7.4)). In the figure, the control (lacking an organic acid) formulations are denoted by circles, with open circles corresponding to the formulation with an exendin-4 concentration of 2 mg/mL and closed circles to the formulation with exendin-4 concentration of 0.4 mg/mL, and the organic-acid containing formulations are denoted by squares, with open squares corresponding to the formulation with an exendin-4 concentration of 2 mg/mL and closed squares to the formulation with exendin-4 concentration of 0.4 mg/mL.

FIG. 3 plots apparent first-order rate decomposition rate constants (extracted from 30-60 day release data) as a function of pH for aqueous suspensions of the incretin mimetic peptide exendin-4 and an organic acid compound as listed in Table 1; data points corresponding to a formulation with a potency retention >70% after 30 days are marked as diamonds and data points corresponding to a formulation with a potency retention <70% after 30 days are marked as open squares.

FIGS. 4A-4B are illustrations of a drug delivery device, in assembled form (FIG. 4A) and in unassembled form (FIG. 4B).

FIG. 5 is a graph of cumulative release of exendin-4, in micrograms, as a function of time, in days, from drug delivery devices comprising an aqueous suspension of the incretin mimetic exendin-4 and an organic acid compound p-aminobenzoic acid (closed squares), p-coumaric acid (triangles), or terephthalic acid (x symbols). Release of exendin-4 from control devices with exendin-4 in PBS lacking an organic acid is also shown (diamonds).

### BRIEF DESCRIPTION OF THE SEQUENCES

SEQ ID NO: 1 corresponds to the amino acid sequence of the peptide compound referred to in the art as GLP-1 [7-37]: HAEGTFTSDV SSYLEGQAAK EFIAWLVKGRG

SEQ ID NO: 2 corresponds to the amino acid sequence of the peptide compound known as exendin-3: His Sec Asp Gly Thr Phe Thr Ser Asp Leu Sec Lys Gln Met Glu Glu Ala Val Arg Leu Phe Ile Glu Trp Leu. Lys Asn Gly Gly Pro Ser Ser Gly Ala Pro Pro Pro Ser

SEQ ID NO: 3 corresponds to the amino acid sequence of the peptide compound known as exendin-4: His Gly Glu Gly Thr Phe Thr Ser Asp Leu Ser Lys Gln Met Glu Glu Glu Ala Val Arg Leu Phe Ile Glu Trp Leu Lys Asn Gly Gly Pro Ser Ser Gly AlaPro Pro Pro Ser

SEQ ID NO: 4 corresponds to an amino acid sequence of a peptide compound that is an analog of exendin-4, referred to as exendin-4 (1-30): His Gly Glu Gly Thr Phe Thr Ser Asp Leu Ser Lys Gln Met Glu Glu Glu Ala Val Arg Leu Phe Ile Glu Trp Leu Lys Asn Gly Gly

SEQ ID NO: 5 corresponds to an amino acid sequence of a peptide compound that is an analog of exendin-4, referred to as ¹⁴Leu, ²⁵Phe exendin-4: His Gly Glu Gly Thr Phe Thr Ser Asp Leu Ser Lys Gln Leu Glu Glu Glu Ala Val Arg Leu Phe Ile Glu Phe Leu Lys Asn Gly Gly Pro Ser Ser Gly Ala Pro Pro Pro Ser

SEQ ID NO: 6 corresponds to an amino acid sequence of a peptide compound that is an analog of exendin-4, referred to as ⁴Leu, ²²Ala, ²⁵Phe exendin-4 (1-28): His Gly Glu Gly Thr Phe Thr Ser Asp Leu Ser Lys Gln Leu Glu Glu Glu Ala Val Arg Leu Ala Ile Glu Phe Leu Lys Asn.

### DETAILED DESCRIPTION

### I. Definitions

Various aspects now will be described more fully hereinafter. Such aspects may, however, be embodied in many different forms and should not be construed as limited to the embodiments set forth herein; rather, these embodiments are provided so that this disclosure will be thorough and complete, and will fully convey its scope to those skilled in the art.

Where a range of values is provided, it is intended that each intervening value between the upper and lower limit of that range and any other stated or intervening value in that stated range is encompassed within the disclosure. For example, if a range of 1 μm to 8 μm is stated, it is intended that 2 μm, 3 μm, 4 μm, 5 μm, 6 μm, and 7 μm are also explicitly disclosed, as well as the range of values greater than or equal to 1 μm and the range of values less than or equal to 8 μm.

The singular forms “a,” “an,” and “the” include plural referents unless the context clearly dictates otherwise. Thus, for example, reference to a “polymer” includes a single polymer as well as two or more of the same or different polymers, reference to an “excipient” includes a single excipient as well as two or more of the same or different excipients, and the like.

The word “about” when immediately preceding a numerical value means a range of plus or minus 10% of that value, e.g., “about 50” means 45 to 55, “about 25,000” means 22,500 to 27,500, etc., unless the context of the disclosure indicates otherwise, or is inconsistent with such an interpretation. For example in a list of numerical values such as “about 49, about 50, about 55,” “about 50” means a range extending to less than half the interval(s) between the preceding and subsequent values, e.g., more than 49.5 to less than 52.5. Furthermore, the phrases “less than about” a value or “greater than about” a value should be understood in view of the definition of the term “about” provided herein.

The compositions of the present disclosure can comprise, consist essentially of, or consist of, the components disclosed.

All percentages, parts and ratios are based upon the total weight of the compositions and all measurements made are at about 25° C., unless otherwise specified.

The term “amino acid” refers to natural amino acids, unnatural amino acids, and amino acid analogs, all in their D and L stereoisomers if their structure allows such stereoisomeric forms. Natural amino acids include alanine (Ala), arginine (Arg), asparagine (Asn), aspartic acid (Asp), cysteine (Cys), glutamine (Gln), glutamic acid (Glu), glycine (Gly), histidine (His), isoleucine (Ile), leucine (Leu), Lysine (Lys), methionine (Met), phenylalanine (Phe), proline (Pro), serine (Ser), threonine (Thr), typtophan (Trp), tyrosine (Tyr) and valine (Val). Unnatural amino acids include, but are not limited to azetidinecarboxylic acid, 2-aminoadipic acid, 3-aminoadipic acid, beta-alanine, aminopropionic acid, 2-aminobutyric acid, 4-aminobutyric acid, 6-aminocaproic acid, 2-aminoheptanoic acid, 2-aminoisobutyric acid, 3-aminoisobutyric acid, 2-aminopimelic acid, tertiary-butylglycine, 2,4-diaminoisobutyric acid, desmosine, 2,2′-diaminopimelic acid, 2,3-diaminopropionic acid, N-ethylglycine, N-ethylasparagine, homoproline, hydroxylysine, allo-hydroxylysine, 3-hydroxyproline, 4-hydroxyproline, isodesmosine, allo-isoleucine, N-methylalanine, N-methylglycine, N-methylisoleucine, N-methylpentylglycine, N-methylvaline, naphthalanine, norvaline, norleucine, ornithine, pentylglycine, pipecolic acid and thioproline. Amino acid analogs include the natural and unnatural amino acids which are chemically blocked, reversibly or irreversibly, or modified on their N-terminal amino group or their side-chain groups, as for example, methionine sulfoxide, methionine sulfone, S-(carboxymethyl)-cysteine, 5-(carboxymethyl)-cysteine sulfoxide and S-(carboxymethyl)-cysteine sulfone.

The term “amino acid analog” refers to an amino acid wherein either the C-terminal carboxy group, the N-terminal amino group or side-chain functional group has been chemically modified to another functional group. For example, aspartic acid-(beta-methyl ester) is an amino acid analog of aspartic acid; N-ethylglycine is an amino acid analog of glycine; or alanine carboxamide is an amino acid analog of alanine.

The terms “peptide,” “polypeptide” and/or “peptide compound” refer to polymers of up to about 80 amino acid residues bound together by amide (CONH) linkages. Analogs, derivatives, agonists, antagonists and pharmaceutically acceptable salts of any of the peptide compounds disclosed here are included in these terms. The terms also include peptides and/or peptide compounds that have D-amino acids, modified, derivatized or naturally occurring amino acids in the D- or L-configuration and/or peptidomimetic units as part of their structure.

The phrase “pharmaceutically acceptable” is employed herein to refer to those compounds, salts, compositions, dosage forms, etc., which are—within the scope of sound medical judgment—suitable for use in contact with the tissues of human beings and/or other mammals without excessive toxicity, irritation, allergic response, or other problem or complication, commensurate with a reasonable benefit/risk ratio. In some aspects, “pharmaceutically acceptable” means approved by a regulatory agency of the federal or a state government, or listed in the U.S. Pharmacopeia or other generally recognized pharmacopeia for use in mammals (e.g., animals), and more particularly, in humans.

The term “chemical stability” means that with respect to the therapeutic agent, an acceptable percentage of degradation products produced by chemical pathways such as oxidation, hydrolysis, or aspartamide formation is formed within a period corresponding to the formulation preparation, storage, distribution, and/or therapeutic dosing. A formulation is considered chemically stable if no more than about 20% of the initial mass of therapeutic agent is lost through one or more chemical processes after one year of storage at the intended storage temperature of the product (e.g., between −20° C. and room temperature); or storage of the product at 30° C./60% relative humidity for one year; or storage of the product at 40° C./75% relative humidity for one month, and preferably three months. In some embodiments, a chemically stable formulation has less than 20%, less than 15%, less than 10%, less than 5%, less than 4%, less than 3%, less than 2%, or less than 1% breakdown products formed on a per mole basis after an extended period of storage at the intended storage temperature of the product. Within other environments of use, a formulation is considered chemically stable if no more than about 30% of the initial mass of therapeutic agent is lost through one or more chemical processes after 30 days, or about 40% of the initial mass of therapeutic agent is lost through one or more chemical processes after 60 days.

The term “physical stability” means that with respect to the therapeutic agent, an acceptably low percentage of insoluble or irreversibly denatured aggregates (e.g., dimers, trimers and larger forms) is formed within a specified time frame. In particular, a formulation is considered physically stable if no more that about 15% of insoluble or irreversibly denatured aggregates are formed from an initial mass of therapeutic agent after one year of storage at the intended storage temperature of the product (e.g., between −20° C. and room temperature); or storage of the product at 30° C./60% relative humidity for one year; or storage of the product at 40° C./75% relative humidity for one month, and preferably three months. In some embodiments, a physically stable formulation has less than 15%, less than 10%, less than 5%, less than 4%, less than 3%, less than 2%, or less than 1% of insoluble or irreversibly denatured aggregates formed from an initial mass of therapeutic agent after an extended period of storage at the intended storage temperature of the product. Within its environment of use, a formulation is considered physically stable if no more than about 30% of the initial mass of therapeutic agent is lost through a precipitation or irreversible denaturation process after 30 days, or about 40% of the initial mass of therapeutic agent is lost through a precipitation or irreversible denaturation process after 60 days. Physically stable formulations may include those that contain a therapeutic agent in an aggregated state, provided that the aggregate in question is soluble and can reversibly and quantitatively generate therapeutically active agent upon dilution; for instance, within a physiological fluid.

The term “stable formulation” means that at least about x % of an initial mass of therapeutic agent remains chemically and physically stable after 60 days of storage at room temperature (25° C.). Particularly preferred formulations are those such that the mass percent of active therapeutic agent remaining after 60 days, defined as x, can be 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% of the initial content of therapeutic agent under these storage conditions.

The term “treating” is used herein in reference to methods of administration of a peptide or peptide compound which reduces the frequency of, or delays the onset of, symptoms of a medical condition (e.g., diabetes, obesity) in a subject relative to a subject not receiving the compound or composition. This can include reversing, reducing, or arresting the symptoms, clinical signs, and underlying pathology of a condition in a manner to improve or stabilize a subject's condition (e.g., controlling diabetes or ameliorating or reversing weight gain).

By reserving the right to proviso out or exclude any individual members of any such group, including any sub-ranges or combinations of sub-ranges within the group, that can be claimed according to a range or in any similar manner, less than the full measure of this disclosure can be claimed for any reason. Further, by reserving the right to proviso out or exclude any individual substituents, analogs, compounds, ligands, structures, or groups thereof, or any members of a claimed group, less than the full measure of this disclosure can be claimed for any reason.

Throughout this disclosure, various patents, patent applications and publications are referenced. The disclosures of these patents, patent applications and publications in their entireties are incorporated into this disclosure by reference in order to more fully describe the state of the art as known to those skilled therein as of the date of this disclosure. This disclosure will govern in the instance that there is any inconsistency between the patents, patent applications and publications cited and this disclosure.

For convenience, certain terms employed in the specification, examples and claims are collected here. Unless defined otherwise, all technical and scientific terms used in this disclosure have the same meanings as commonly understood by one of ordinary skill in the art to which this disclosure belongs.

### II. Formulations to Stabilize an Incretin Mimetic

A composition or formulation in which an incretin mimetic compound is chemically stable, physically stable, or both is provided. Because of the stability of the incretin mimetic in the formulation, the incretin mimetic is deliverable from a device or drug delivery platform for a sustained period of time. In one embodiment, the composition is an aqueous suspension. In another embodiment, the composition is a heterogeneous or nonuniform mixture, solution, or slurry. The solution or mixture can be, in some embodiments, an aqueous mixture or an aqueous heterogeneous mixture. In another embodiment, the composition is in dry form (e.g, lyophilized, spray dried, desiccated, freeze-dried, etc.). In these various embodiments, the composition comprises an incretin mimetic and an organic acid that has one or more of these features: (i) has a water solubility at room temperature (e.g., approximately 25° C.) of less than about 20 g/L or of between about 0.01 and 10 g/L, (ii) has a molar mass equal to or less than about 500 grams per mole, and (iii) maintains a pH of the suspension (or solution) in its environment of use of between 3.0-6.0 for a period of at least about 30 days. The composition may additionally comprise an aqueous fluid, for example water, buffer or a water-solvent mixture. In embodiments where the composition is in dry form, the aqueous fluid hydrates the composition in situ in its environment of use.

As noted above, the formulations described herein provide chemical and/or physical stability of the incretin mimetic in order to permit delivery for a sustained period. In one embodiment, a sustained period of time intends a period of at least about two weeks to about six months. In another embodiment, a sustained period of time intends a period of at least about two weeks, or at least about three weeks, or at least about four weeks to about six months, or to about four months, or to about three months. In another embodiment, a sustained period of time intends a period of at least about 15 days, or at least about 21 days, or at least about 30 days, or at least about 45 days, or at least about 60 days.

Also as noted above, the formulations described herein provide the described stability of the incretin mimetic in part by maintaining a particular pH range of the formulation in its environment of use for the stated period of time. In one embodiment, the environment of use is in vivo. For example, the formulation may be part of a drug delivery device that is implanted in vivo and several examples of such devices are provided below. In another embodiment, the environment of use is in vitro in a release medium maintained at about 37° C.

The components of the composition, namely the incretin mimetic and the organic acid, are now described.

### A. Incretin Mimetic Compounds

The formulations comprise an incretin mimetic. Incretin mimetics are agents that act like incretin hormones, binding receptors for glucagon-like peptide-1 (GLP-1). Drugs in the class of incretin mimetics are known in the art and include, for example, exenatide (sold under the trade names BYETTA® and BYDUREON®) and liraglutide (sold under the trade name VICTOZA®). These compounds work by mimicking the incretin hormones that the body usually produces naturally to stimulate the release of insulin in response to a meal.

In one embodiment, the incretin mimetic is a glucagon-like peptide-1 (GLP-1) agonist. The term “GLP-1 agonist” as used herein refers to a compound which fully or partially activates the human GLP-1 receptor. In some embodiments the “GLP-1 agonist” binds to a GLP-1 receptor, e.g., with an affinity constant (K D) or activates the receptor with a potency (EC₅₀) of below 1 μM, e.g. below 100 nM as measured by methods known in the art (see e.g. WO 98/08871) and exhibits insulinotropic activity, where insulinotropic activity may be measured in vivo or in vitro using assays known to those of ordinary skill in the art. For example, the GLP-1 agonist may be administered to an animal with increased blood glucose (e.g. obtained using an Intravenous Glucose Tolerance Test (IVGTT)) and the plasma insulin concentration measured over time.

Examples of GLP-1 agonists are known in the art (see, for example, U.S. Pat. Nos. 5,424,286; 6,858,576; 6,872,700; 6,902,744; 6,956,026; 7,297,761; 7,521,423; 7,741,269; 8,329,648; 8,431,685; 8,906,851; 9,238,076; 7,612,176; 7,563,871; 7,456,254; 7,223,440; 6,824,822; 6,667,061; 6,495,164; 6,479,065; 6,458,924 and 9,265,723, which are each incorporated by reference herein). Several examples are set forth herein to illustrate the compounds. In a first embodiment, the GLP-1 agonist compound is identical to or having a certain sequence identity to GLP-1 (7-37), identified herein as SEQ ID NO:1. In one embodiment the GLP-1 agonist exhibits at least 60%, 65%, 70%, 80%, 81%, 82%, 83%, 84%, 85%, 86%, 87%, 88%, 89%, 90%, 91%, 92%, 93%, 94%, 95%, 96%, 97%, 98% or 99% sequence identity to GLP-1(7-37) over the entire length of GLP-1(7-37). As an example of a method for determination of sequence identity between two peptides, the peptide GLP-1(7-37) (SEQ ID NO: 1) and a peptide known in the art as [Aib8]GLP-1(7-37) in which the Ala residue in position 8 has been substituted with 2-aminoisobutyric acid (Aib) are aligned. Sequence identity is given by the number of aligned identical residues minus the number of different residues divided by the total number of residues in GLP-1(7-37). Accordingly, in this example the sequence identity is (31-1)/31 or 98%.

In another embodiment, the GLP-1 agonist is an exendin or an exendin-analogue. Exendins are a family of peptides that have amino acid sequence similarity to several members of the glucagon-like peptide family and are known to be potent GLP-1 receptor agonists, Exendin agonist compounds are described, for example, in U.S. Pat. Nos. 6,872,700 and 6,956,026, and other patent documents references above and incorporated by reference herein. One example is the peptide known as exendin-4, which is identified herein as SEQ ID NO: 3. Exendin-4 is a potent GLP-1 receptor agonist that stimulates somatostatin release and inhibits gastrin release (Goke, et al., J. Biol. Chem. 68:19650-55, 1993 Schepp, et al., Eur. J. Pharmacol., 69:183-91, 1994; Eissele, et al., Life Sci., 55:629-34, 1994), Another example is exendin-3, which is identified herein as SEQ ID NO: 2. Exendin-3 and exendin-4 are GLP-1 receptor agonists and are used for the treatment of diabetes mellitus, for reduction of gastric motility, for treating obesity, and other uses.

In general, the GLP-1 agonist for use in the compositions described herein is an analogue of GLP-1 (SEQ ID NO: 1) or of an exendin peptide (e.g., SEQ ID NO; 2 and SEQ ID NO: 3), optionally comprising a substituent. The term “analogue” means a peptide wherein at least one amino acid residue of the peptide has been substituted with another amino acid residue and/or wherein at least one amino acid residue has been deleted from the peptide and/or wherein at least one amino acid residue has been added to the peptide and/or wherein at least one amino acid residue of the peptide has been modified. Such addition or deletion of amino acid residues may take place at the N-terminus of the peptide and/or at the C-terminus of the peptide. GLP-1 analogues may have substitutions with a naturally occurring amino acid (e.g., one of the 21 proteinogenic amino acids) or with a non-proteinogenic amino acid, such as or 2-aminoisobutyric acid (Aib).

By way of further example, the GLP-1 agonist analogue, [Aib8] GLP-1(7-37) designates an analogue of GLP-1(7-37) wherein the naturally occurring Ala in position 8 has been substituted with Aib. Another example is the GLP-1 agonist ¹⁴Leu, ²⁵Phe exendin-4 in which the amino acid residues at positions 14 and 25 of the GLP-1 agonist exendin-4 (SEQ ID NO: 3) are substituted with a leucine and with phenylalanine, respectively. The sequence of ¹⁴Leu, ²⁵Phe exendin-4 is set forth herein as SEQ ID NO: 5. Another example of a GLP-1 agonist analogue is set forth at SEQ ID NO: 6, which is another analog of exendin-4, referred to as ⁴Leu, ²²Ala, ²⁵Phe exendin-4 (1-28).

In some embodiments, the GLP-1 agonist comprises a maximum of twelve, such as a maximum of 10, 8 or 6, amino acids which have been altered, e.g., by substitution, deletion, insertion and/or modification, compared to e.g. GLP-1(7-37). In some embodiments the analogue comprises up to 10 substitutions, deletions, additions and/or insertions, such as up to 9 substitutions, deletions, additions and/or insertions, up to 8 substitutions, deletions, additions and/or insertions, up to 7 substitutions, deletions, additions and/or insertions, up to 6 substitutions, deletions, additions and/or insertions, up to 5 substitutions, deletions, additions and/or insertions, up to 4 substitutions, deletions, additions and/or insertions or up to 3 substitutions, deletions, additions and/or insertions, compared to e.g. GLP-1(7-37). Unless otherwise stated the GLP-1 comprises only L-amino acids.

In another embodiment, the GLP-1 agonist is GLP-1(7-37); GLP-1(7-36)NH₂; exendin-3; exendin-4; exendin-4 analogues and amidated exendin-4 analogues, in which one or more amino acid residues have been replaced by different amino acid residues including N-terminal modifications; truncated exendin-4 and truncated forms that are amidated; truncated exendin-3 and truncated forms that are amidated, or the compounds known as AVE-0010(ZP-10) (Sanofi-Aventis Zealand Pharma), BAY-73-7977 (Bayer), TH-0318, BIM-51077 (Ipsen, Tejin, Roche), N,N-2211 (Novo Nordisk), LY315902.

It will be appreciated that the GLP-1 agonist compound can be modified with polyethylene glycol, as described for example in U.S. Pat. No. 6,872,700. The GLP-agonist peptide may be linked to one or more polyethylene glycol polymers, or other molecular weight enhancing molecules. The polyethylene glycol polymers may have molecular weights between 500 Daltons and 20,000 Daltons. The polyethylene glycol polymers are preferably linked to an amino, carboxyl, or thio group, and may be linked to the N or C termini of the peptide, or to the side chains of lysine, aspartic acid, glutamic acid, or cysteine, or alternatively, the polyethylene glycol polymers may be linked with diamine and dicarboxylic groups. In one embodiment, the GLP-1 agonist is an exendin or exendin agonist linked to the polyethylene glycol polymers through an epsilon amino group on a lysine amino acid of the exendin or exendin agonist.

In another embodiment, the incretin mimetic is selected from the group consisting of albiglutide, dulaglutide, and liraglutide. Albiglutide is a GLP-1 receptor agonist in the form of a recombinant fusion protein comprised of 2 copies of modified human GLP-1 genetically fused in tandem to human albumin. The human GLP-1 fragment sequence 7-36 has been modified with a glycine substituted for the naturally-occurring alanine at position 8 in order to confer resistance to dipeptidylpeptidase IV (DPP-IV) mediated proteolysis. The human albumin moiety of the recombinant fusion protein, together with the DPP-IV resistance, extends the half-life allowing once-weekly dosing. The protein is sold under the tradename TANZEUM®. See, U.S. Pat. Nos. 8,809,271; 8,759,284, 8,969,293; which are incorporated by reference herein.

Dulaglutide is a GLP-1 receptor agonist sold under the brand name TRULICITY®, as an adjunct to diet and exercise to improve glycemic control in adults with type 2 diabetes mellitus. The recommended dosing is 0.75 mg once weekly, which may be increased to the maximum recommended dose of 1.5 mg once weekly, given by subcutaneous injection. The protein is a fusion protein that consists of 2 identical, disulfide-linked chains, each containing an N-terminal GLP-1 analog sequence covalently linked to the Fc portion of a modified human immunoglobulin G4 (IgG4) heavy chain by a small peptide linker and is produced using mammalian cell culture. The GLP-1 analog portion of dulaglutide is 90% homologous to native human GLP-1 (7-37) (identified herein as SEQ ID NO: 1). Structural modifications were introduced in the GLP-1 part of the molecule responsible for interaction with the enzyme dipeptidyl-peptidase IV (DPP-4). Additional modifications were made in an area with a potential T-cell epitope and in the areas of the IgG4 Fc part of the molecule responsible for binding the high-affinity Fc receptors and half-antibody formation. The overall molecular weight of dulaglutide is approximately 63 kilodaltons.

Liraglutide is a glucagon-like peptide-1 receptor agonist peptide compound marketed under the brand name SAXENDA® for the treatment of type 2 diabetes and for the treatment of obese or overweight adults with at least one weight-related comorbid condition. The peptide known as liraglutide is comprised of a fatty acid molecule attached at one position of the GLP-1-(7-37) molecule, enabling it to both self-associate and bind to albumin within the subcutaneous tissue and bloodstream. The active GLP-1 is released from albumin. See, U.S. Pat. Nos. 9,132,239; 9,108,002; 8,920,383; 8,846,618; 8,809,271; 8,684,969; 8,114,833; 8,809,271 7,686,786; 7,235,627; 6,899,699; 6,458,924; 6,268,343; each incorporated by reference herein.

The incretin mimetic compounds in the compositions described herein are present in an amount to provide therapy for the intended period of time. Therapeutically effective amounts of the compounds, particularly of an exendin, exendin agonist, or modified exendin, are known in the literature and from the package inserts. As will be recognized by those in the field, an effective amount of therapeutic agent will vary with many factors including the age and weight of the patient, the patient's physical condition, the glucagon level or level of inhibition of glucagon suppression to be obtained, and other factors. By way of example, an effective daily dose for some of the compounds will typically be in the range of 0.01 or 0.03 to about 5 mg/day, preferably about 0.01 or 0.5 to 2 mg/day and more preferably about 0.01 or 0.1 to 1 mg/day, for a 70 kg patient. The exact dose to be administered is determined by the attending clinician and is dependent upon where the particular compound lies within the above quoted range, as well as upon the age, weight and condition of the individual.

### B. Organic Acids

The composition, in addition to an incretin mimetic compound, comprises an organic acid. The organic acid is one that (i) has a water solubility at room temperature (20-25° C.) of less than about 20 g/L or between about 0.01 and 10 g/L, (ii) a molar mass of less than or equal to 500 grams per mole, and/or (iii) maintains a pH of the suspension or solution in its environment of use of between 3.0-6.0 for a period of at least about 30 days. Reference herein to “an organic acid compound” or a “stabilizing organic acid” or an organic acid with limited water solubility” intends an organic acid is one that (i) has a water solubility at room temperature of less than about 10 g/L and (ii) maintains a pH of the suspension or solution in its environment of use of between 3.0-6.0 for a period of at least about 30 days. In another embodiment, the organic acid can be one that has a water solubility at room temperature of less than about 18 g/L, 15 g/L, 12 g/L, 10 g/L, 8 g/L or 5 g/L.

As described above, the compositions provide chemical stability, physical stability, or both to the incretin mimetic compound, permitting use of the composition in a drug delivery platform that provides sustained release for an extended period of time. Examples of organic acids for use in the compositions are now described.

In a first embodiment, the organic acid is a carboxylic acid. Examples include aromatic carboxylic acids where a carboxylic acid group is bonded directly to an aromatic ring. For example, the aromatic carboxylic acid can have one carboxylic acid group bound to an unsubstituted benzene or pyridine ring. Examples include benzoic acid, picolinic acid, nicotinic acid, or isonicotinic acid. In another example, the aromatic carboxylic acid is one having a benzene ring and one electron-donating group with antioxidant properties. Specific examples include o-anisic acid, m-anisic acid, p-anisic acid, p-aminobenzoic acid (PABA), o-aminobenzoic acid (anthranilic acid), o-toluic acid, m-toluic acid, p-toluic acid and salicylic acid.

In yet another example, the aromatic carboxylic acid is one having a single benzene ring and two electron donating groups with antioxidant properties. A specific example is vanillic acid. In still another example, the aromatic carboxylic acid is one having two or more carboxylic acid groups bonded to a benzene ring. A specific example is terephthalic acid.

In another example, the aromatic carboxylic acid is one having one carboxylic acid group bonded to a naphthalene or quinoline ring. Examples include 1-naphthoic acid, 2-naphthoic acid, quinaldic acid, 3-quinolinecarboxylic acid, 4-quinolinecarboxylic acid, 5-quinolinecarboxylic acid, 6-quinolinecarboxylic acid, 7-quinolinecarboxylic acid, and 8-quinolinecarboxylic acid. A further grouping of acids of this type, with one carboxylic acid group bonded to a naphthalene or quinoline ring, include those containing an additional electron-donating group, such as a hydroxy, methoxy, amino, alkylamino, dialkylamino, or alkyl group. Examples of acids in this grouping include 6-hydroxy-2-naphthoic acid, 6-hydroxy-3-naphthoic acid, 8-hydroxy-2-quinolinecarboxylic acid, 8-hydroxy-7-quinolinecarboxylic acid, and isomers of each.

In another exemplary embodiment, the carboxylic acid is one having a carboxylic acid group bonded to a biphenyl ring system that bears a hydroxyl group or other electron donating substituent. Examples include 4′-hydroxy-4-biphenylcarboxylic acid, 4′-hydroxy-2-biphenylcarboxylicacid, 4′-methyl-4-biphenyl c arb oxylic acid, 4′-methyl-2-biphenyl carb oxylic acid, 4′-methoxy-4-biphenylcarboxylic acid, and 4′-methoxy-2-biphenylcarboxylic acid.

In another exemplary embodiment, the carboxylic acid is one having one or two carboxylic acid groups directly bonded to a biphenyl ring system. Examples include 2-phenylbenzoic acid, 3-phenylbenzoic acid, 4-phenylbenzoic acid and diphenic acid.

In another exemplary embodiment, the carboxylic acid is one having a carboxylic acid functional group separated from a benzene, pyridine, naphthalene, or quinoline ring by a chain of 1-4 saturated carbon atoms. Examples of acids in this embodiment include phenylacetic acid and 3-phenylpropionic acid.

In another exemplary embodiment, the carboxylic acid is an aliphatic dicarboxylic acid with 6-10 carbon atoms, such as adipic acid ((CH₂)₄(COOH)₂), pimelic acid (HO₂C(CH₂)₅CO₂H), suberic acid (HO₂C(CH₂)₆CO₂H), azelaic acid (HO₂C(CH₂)₇CO₂H), and sebacic acid (HO₂C(CH₂)₈CO₂H).

In another exemplary embodiment, the carboxylic acid is an unsaturated or polyunsaturated dicarboxylic acid containing 4-10 carbons. Examples of acids in this embodiment include fumaric acid, trans,trans-muconic acid, cis, trans-muconic acid, and cis,cis-muconic acid.

In another exemplary embodiment, the carboxylic acid is a cis-or trans-cinnamic acid. In one embodiment, the trans-cinnamic acid has one or two electron-donating groups selected from hydroxy, methoxy, amino, alkylamino, dialkylamino, or alkyl groups. Examples include o-coumaric acid, m-coumaric acid, p-coumaric acid, o-methylcinnamic acid, m-methylcinnamic acid, p-methylcinnamic acid, o-methoxycinnamic acid, m-methoxycinnamic acid, p-methoxycinnamic acid, and ferulic acid.

In another embodiment, the organic acid is a phenol or a naphthol substituted with between about 2-5 electron-withdrawing groups selected from F, Cl, Br, I, CN, CHO and NO₂. Examples include pentafluorophenol or 2,4-dinitrophenol.

In another embodiment, the organic acid is a 1,3-dicarbonyl compound containing an acidic CH bond (pKa<8). Examples include 2,2-dimethyl-1,3-dioxane-4,6-dione (Meldrum's acid), cyanuric acid, or barbituric acid.

In another embodiment, the organic acid is an imide, such as phthalimide. In one embodiment, the phthalimide is substituted with at least one electron-withdrawing substituent.

In another embodiment, the organic acid is a hydroxamic acid. The hydroxamic acid may be, in some embodiments, an aromatic hydroxamic acid containing one hydroxamic functional group bonded directly to an aromatic ring. The aromatic ring is selected from the group consisting of a benzene ring, a pyridine ring, a naphthalene ring, a quinoline ring, and a biphenyl ring. Examples include benzhydroxamic acid. The hydroxamic acid can also be one containing a hydroxamic functional group separated from an aromatic ring by a chain of 1-4 sp³-hybridized carbon atoms. Dihydroxamic acids containing two or more hydroxamic acid functional groups bonded directly to a benzene, pyridine, naphthalene, quinoline, or biphenyl ring system are also contemplated. In addition, substituted derivatives of the hydroxamic acids described above that contain electron donating substituents such as hydroxy, methoxy, amino, alkylamino, dialkylamino, or alkyl groups are contemplated. Also contemplated are aliphatic dihydroxamic acids containing 6-10 carbon atoms, such as suberohydroxamic acid, and unsaturated dihydroxamic acids containing 6-10 carbon atoms.

The organic acids for use in the compositions described herein are those with a water solubility at room temperature of less than about 20 g/L or less than 10 g/L. In another embodiment, the acid has a water solubility at room temperature of between about 0.01 and 10 g/L, a molar mass of 500 gram per mole or less, and/or a pKa value between 3 and 6. In other embodiments, the organic acid is crystalline and has a melting temperature of more than about 37° C. In another embodiment, the organic acids for use in the compositions described herein are non-polymeric or non-oligomeric. In another embodiment, the organic acids for use in the compositions described herein do not have a polymeric or oligomeric backbone and/or are not attached to a polymeric or oligomeric backbone.

Compositions comprising an organic acid and an incretin mimetic compound are prepared by mixing the organic acid and the incretin mimetic together in a suitable solvent. In some embodiments, the solvent is an aqueous fluid, such as a buffer or a water-organic solvent mixture. The organic acid is present in the composition at any concentration, yet in a preferred embodiment is present in an amount such that the organic acid is at or above its saturation concentration in the composition. In a preferred embodiment, the organic acid is present in an amount such that at the end of the delivery period, it remains at or above its saturation concentration within its environment of use.

A study was conducted to evaluate a variety of organic acids for use in the compositions described herein. As detailed in Example 1, exendin-4 (SEQ ID NO: 3) was dissolved into phosphate buffered saline (2 mg/mL; initial pH=7.4) and combined with partially soluble acids at a concentration greater than their saturation point (˜20 mg solid/mL peptide solution). The acids in the compositions included p-coumaric acid, m-coumaric acid, p-methoxycinnamic acid, trans-cinnamic acid, 4-methylcinnamic acid, and 4-aminobenzoic acid (PABA). The pH values of these formulations were obtained, and found to range from approximately 4 to 5.4. Additionally, control formulations were prepared to omit an acid (phosphatebuffer, pH=7.2) or to include a more concentrated, soluble organic acid e.g., citric acid at ≥20 g/L (pH=2.0). The suspensions (formulations) were incubated at 37° C. and stability of the peptide in each suspension was assessed by quantifying the peptide present in aliquots taken at selected time points.

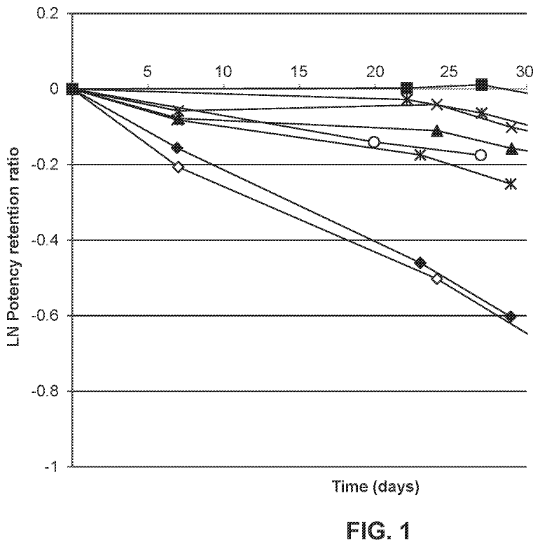

Data obtained over the first four weeks of the study are shown in FIG. 1. The Y-axis shows the log of the potency retention ratio of the peptide, defined as the quantity of intact peptide remaining at each time point divided by the initial peptide load at time zero. The figure shows results for the formulations comprising p-coumaric acid (closed squares), m-coumaric acid (x symbols), p-methoxycinnamic acid (closed triangles), trans-cinnamic acid (open triangles), 4-methylcinnamic acid (asterisks), 4-aminobenzoic acid (PABA) (open circles), citric acid (water soluble acid, as a control, closed diamonds), and PBS control (open diamonds). Although exendin-4 is assumed to degrade by multiple mechanisms, the decay kinetics of acidic formulations appears to be well-approximated by a first-order model for most of the examined formulations for a period of at least 30 days post-constitution. The negative slopes of the lines correspond to approximate first order rate constants, with steeper lines corresponding to less stable formulations. The formulations (suspensions) with pH values between approximately 4.0 to 5.4 displayed enhanced stability of the peptide relative to a formulation lacking the organic acid, i.e., the phosphate buffered saline control formulation (pH-7.2), or to a formulation containing a water soluble organic acid at a high concentration; e.g., 20 mg/mL citric acid; pH-2.0.

In another study, described in Example 2, the stability of a peptide incretin mimetic in a formulation with an organic acid was evaluated. Formulations were prepared to either include or exclude a stabilizing organic acid (p-coumaric acid, nominally at ˜20 mg/mL to greatly exceed its saturation point) in phosphate-buffered saline, at two distinct concentrations of exendin-4 (approximately 2.0 mg/mL or 0.4 mg/mL). The formulations were incubated at 37° C. and aliquots of the aqueous fraction were taken at selected time points for analysis of peptide stability.

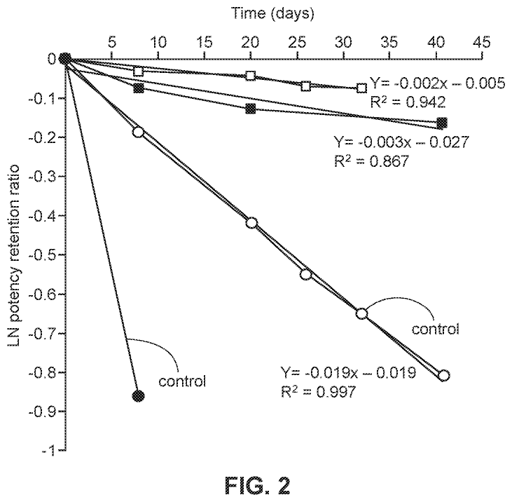

FIG. 2 fits the acquired data to a first-order kinetic model over the first 30-40 days of the study. The Y-axis shows the log of the potency retention ratio of the peptide (defined as the quantity of intact peptide remaining at each time point divided by the initial peptide load at time zero). In the figure, the control (lacking an organic acid) formulations are denoted by circles, with open circles corresponding to the formulation with an exendin-4 concentration of 2 mg/mL and closed circles to the formulation with exendin-4 concentration of 0.4 mg/mL, and the organic-acid containing formulations are denoted by squares, with open squares corresponding to the formulation with an exendin-4 concentration of 2 mg/mL and closed squares to the formulation with exendin-4 concentration of 0.4 mg/mL. The negative slopes of the resulting lines are therefore proportional to apparent first order rate constants, with steeper lines corresponding to less stable formulations. These results show that more concentrated formulations are more stable than less concentrated ones, but that the effect is greatly attenuated at a pH close to the isoelectric point of exendin-4; i.e., a pH that should maximize the formation of soluble aggregates at all concentrations.

In another study, compositions were prepared with the organic acids listed in Table 1.

TABLE 1

| | | Pseudo- | Est. | |
|---|---|---|---|---|
| | Formulation | first | Solubility, | |
| Formulation | pH | order k | 25° C. (g/L) | pKa |
| Citric | 2.04 | 0.02036 | 592 | 3.13 |
| rac-Mandelic | 2.42 | 0.00806 | 158.7 | 3.85 |
| R-Mandelic | 2.45 | 0.00948 | 158.7 | 3.85 |
| Nicotinic | 3.68 | 0.00547 | 18 | 4.75 |
| m-Coumaric | 3.95 | 0.00297 | 1.04 | 4.01 |
| PABA | 4.21 | 0.00421 | 5.9 | 4.65 |
| trans-Cinnamic | 4.35 | 0.00365 | 0.5 | 4.44 |
| p-Coumaric | 4.36 | 0.00124 | 1-10 | 4.64 |
| m-Methoxycinnamic | 4.49 | −0.00059 | 4.46 | 4.47 |
| 4-Chlorobenzoic | 4.81 | 0.00613 | 0.077 | 3.98 |
| p-Anisic | 5.05 | 0.00537 | 0.4 | 4.34 |
| Terephthalic | 5.31 | 0.00027 | 0.017 | 3.51 |
| p-Methoxycinnamic | 5.37 | 0.00213 | 0.712 | 4.04 |
| Cholic | 5.65 | 0.00851 | 0.05 | 5.07 |
| 4-Methylcinnamic | 6.13 | 0.00798 | | |
| 4-Chlorocinnamic | 6.34 | 0.00706 | | 4.41 |
| Sebacic | 6.61 | 0.00511 | 0.25 | 4.72 |
| Control | 7.40 | 0.01711 | | |

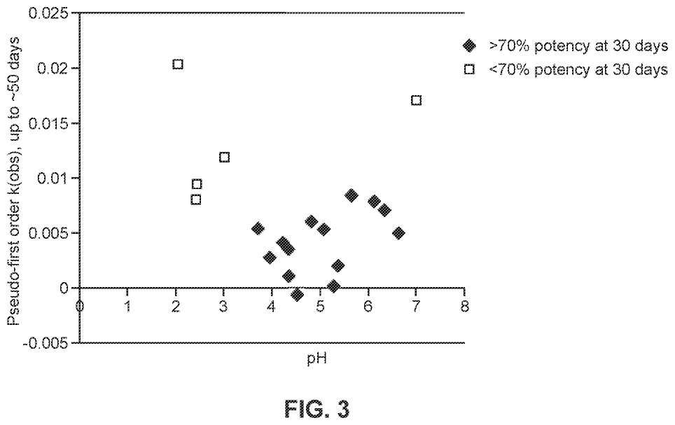

Static compositions comprised of the acids listed in Table 1 with exendin-4 as a model incretin mimetic in phosphate-buffered saline (˜2 mg/mL) were assembled and incubated as described for Example 1. Aliquots were taken at selected time points and analyzed by HPLC to obtain pseudo-first order rate constants describing the decomposition of the incretin mimetic. These rate constants were plotted against formulation pH and the plot is shown in FIG. 3. Points corresponding to formulations showing >70% retention of potency after 30 days are marked as closed diamonds. Points corresponding to formulations showing <70% retention of potency after 30 days are marked as open squares. The results show that the pH of the formulation is a strong predictor of exendin-4 stability, where soluble, stronger acids (e.g., mandelic acid (pH 2.42) and benzilic acid pH (3.05)) elicit very little, if any, stabilizing effect relative to a control, whereas saturated solutions of less soluble, weaker acids that lower the formulation pH to approximately 3-6, or to approximately pH 3.5-6.0 or approximately 4-5.5 provide a stabilizing effect. Formulations with pH values between 4 and 5—i.e., those constructed with organic acids with limited solubility in water (i.e., a water solubility of less than about 10 g/L at room temperature)—stabilized the peptide relative to a control formulation at neutral pH (˜7.2-7.4) as well as to formulations containing ≥20 mg/mL of a soluble, stronger acid (e.g., citric or mandelic acid).

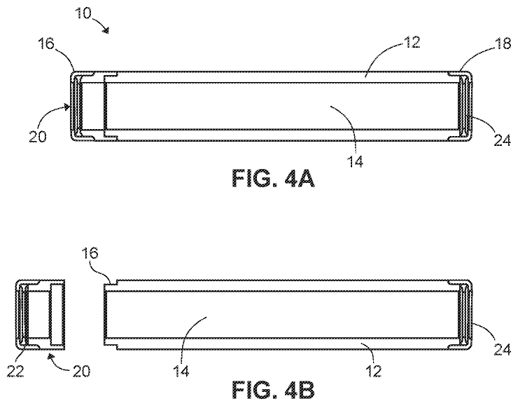

Accordingly, a composition. and a device comprising the composition, are contemplated, where the composition is an aqueous suspension or heterogeneous mixture comprising an incretin mimetic and an organic acid that (i) has water solubility at room temperature of between about 0.01 and 20 g/L, (ii) a molar mass of less than about 500 grams per mole (or in some embodiments equal to or less than 500 grams/mole), and/or (iii) maintains a pH of the suspension or solution in its environment of use of between 3.0-6.0 for a period of at least about 30 days. In one embodiment, the incretin mimetic in the solution or suspension maintains at least about 70% of its potency for the period at 37° C. Examples of a drug delivery device comprising a composition as described herein are described below with reference to FIGS. 4A-4B and Example 4.

The compositions described herein include the organic acid in the form of a suspension or slurry, given its limited water solubility. The organic acid is present in the composition in an amount above its saturation concentration, and, in accord with another embodiment, the organic acid is present in the composition at the end of the delivery period in an amount at or above its saturation concentration. In this way, the composition maintains the desired pH of the suspension or heterogeneous solution of between 3.0-6.0, preferably 2.75-5.75, more preferably 2.8-5.6, preferably 2.9-5.6, preferably 3.1-5.5, 3.2-5.5, 3.3-5.5, 3.4-5.5, 3.5-5.5, 3.1-5.4, 3.2-5.4, 3.3-5.4, 3.4-5.4, 3.5-3.1-5.3, 3.2-5.3, 3.3-5.3, 3.4-5.3, 3.5-5.3, 3.1-5.2, 3.2-5.2, 3.3-5.2, 3.4-5.2, 3.5-5.2, 3.1-5.1, 3.2-3.3-5.1, 3.4-5.1, 3.5-5.1, 3.1-5.0, 3.2-5.0, 3.3-5.0, 3.4-5.0, 3.5-5.0, 3.5-5.5 or 3.5-6.0

In another embodiment, the organic acid is crystalline and has a melting temperature of greater than about or equal to about 37° C. Such organic acids remain in solid form in an in vivo environment of use to provide a heterogeneous mixture or suspension of the organic acid in the composition for the period of delivery time.

As mentioned above, the compositions stabilize the incretin mimetic, thus permitting delivery of a therapeutically active compound for the sustained period of time. In one embodiment, the incretin mimetic is stabilized by the composition such that it retains at least about 70%, 75%, 80%, or 85% of its potency for at least about 30 days, or in another embodiment, for the sustained period of time. Stability intends chemical stability and/or physical stability. In one embodiment, the compositions described herein provide chemical stability of the incretin mimetic. In one embodiment, the compositions described herein provide physical stability of the incretin mimetic. In another embodiment, the compositions described herein provide physical and chemical stability of the incretin mimetic. In one embodiment, the physical and chemical stability of the incretin mimetic is measured in vitro, and intends that the compound resists degradation and/or precipitation to a degree sufficient to maintain its original biological activity in an in vitro environment. Methods for measuring the chemical stability and biological activity of peptides and proteins are well known in the art. For example, circular dichroism (CD) measurements and other methods allow a person skilled in the art to determine the structural properties of a protein or peptide. Other established biophysical methods include nuclear magnetic resonance (NMR) spectroscopy, absorption spectrometry, infrared and Raman spectrometry, mass spectrometry, x-ray crystallography, measurement of the hydrodynamic volume via size exclusion chromatography, analytical ultracentrifugation or dynamic/static light scattering as well as measurements of the frictional coefficient or intrinsic viscosity. More particularly, methods to determine whether a peptide or protein is chemically intact include mass spectrometry and x-ray crystallography. Methods to determine the biological activity of a peptide or protein include a cell-based assay.

In one embodiment, the incretin mimetic is present in the composition at the beginning of the period at a concentration in the solution of greater than or equal to 1 mg/mL. In other embodiments, the incretin mimetic is present in the composition at the beginning of the period at a concentration exceeding 2 mg/mL, or 5 mg/mL, or 10 mg/mL, or 20 mg/mL, or 50 mg/mL. In another embodiment, the incretin mimetic is stable in the composition such that 70% of its initial biological activity is retained for a 30 day period.

### Delivery Device

In another aspect, a drug delivery device for administration of a composition or aqueous suspension as described herein is provided. The drug delivery device can be any implantable device, based on, for example, diffusive, erodible or convective systems, e.g., diffusional systems, osmotic pumps, electro-diffusion systems, electro-osmosis systems, electromechanical systems, and the like. In one embodiment, a controlled drug delivery device is utilized, for controlled, extended delivery of the composition for a period of time. The term “controlled drug delivery device” is meant to encompass any device wherein the release (e.g., rate, timing of release) of drug or other desired substance contained therein is controlled by or determined by the device itself and not the environment of use. Several non-limiting examples are described.

In one embodiment, the drug delivery device is one having a housing member that defines a reservoir in which the compositions and/or the aqueous suspensions described above are retained. The housing member is of a size and shape that is suitable for implantation into the body. A cylindrical shape is preferable for subcutaneous implantation using a cannula or trocar. The outer diameter of a cylindrically shaped housing member would preferably be in the range of 2 mm to 6 mm and the length between about 10 mm-50 mm. The composition or aqueous suspension, in one embodiment, is in a dry form in the reservoir of the device. For example, the aqueous suspension comprising the small molecule therapeutic agent and the organic acid is prepared and is then spray dried, milled or lyophilized to provide a dried form of the aqueous suspension. Alternatively, the individual components in dried form—i.e., the therapeutic agent as a dry solid and the organic acid as a dry solid — are mixed in the correct proportions to provide upon later hydration the desired aqueous suspension. Alternatively, the therapeutic agent and the organic acid may be co-dissolved or suspended within a suitable organic solvent such as methanol, ethanol, 1-propanol, 2-propanol, tert-butanol, acetone, 2-butanone, or ethyl acetate, followed by concentration to yield a dried powder suitable for resuspension into an aqueous medium. The dried form of the composition can be lyophilized, tableted, or pelleted and hydrated in situ upon subcutaneous implantation of a device comprising the dried composition, or can be hydrated at the time of subcutaneous implantation by a clinician introducing a liquid (e.g. a physiological buffer, isotonic saline, or a phosphate buffered saline). The liquid can be provided as part of a kit comprising the drug delivery device and a vial comprising a hydration liquid.

An example of a drug delivery device is provided in FIGS. 4A-4B. FIG. 4A illustrates a device assembled and ready for implantation, in an anatomical compartment of a subject, such as under the skin or in the peritoneal cavity. The device is comprised of a non-erodible housing member 12 that defines an internal compartment or reservoir 14. Contained within the reservoir is a composition or formulation as described herein. Housing member 12 has first and second ends, 16, 18. First end 16 is sealed with a fluid-tight end-cap 20, seen best in FIG. 4B that illustrates device 10 in its unassembled form. End cap 20 may optionally comprise a porous membrane or semi-permeable membrane or porous partition 22. Second end 18 is fitted with a porous membrane, semi-permeable membrane, or porous partition 24.

In one embodiment, the devices and compositions disclosed herein comprise an incretin mimetic and a stoichometric excess of an organic acid that (i) has a solubility in water between about and 10 g/L at 25° C., and (ii) a molar mass of less than 500 grams/mole, and/or (iii) dissolves in the presence of the incretin mimetic and a physiological buffer to produce a suspension or slurry with a pH (within the aqueous fraction) approximately equal to or less than the isoelectric point of the protonated incretin mimetic.

In another embodiment, the devices and compositions disclosed herein comprise an incretin mimetic and a stoichometric excess of an organic acid that (i) has a solubility in water between about and 10 g/L at 25° C., (ii) a molar mass less than 500 grams per mole, and/or (iii) dissolves in the presence of the incretin mimetic and a physiological buffer to produce a suspension or slurry with a pH (within the aqueous fraction) between approximately 3 and 5.

It will be appreciated that a porous partition can be positioned at one or both ends of the housing member. As used herein, the terms “porous membrane” and “porous partition” intend a structural member that has a plurality of pores in the nanometer or micrometer (μm) range, preferably in the 0.1-100 nm or 0.1-200 nm range. The porous partition permits passage of the therapeutic agent in its soluble form from the formulation contained within the reservoir. The porous partition can also permit passage of the organic acid that is part of the formulation in its soluble form. The porous partition in a preferred embodiment retains the therapeutic agent and/or the organic acid in their insoluble forms. That is, the therapeutic agent and/or the organic acid in insoluble form preferably do not pass through the pores of the porous partition. The drug delivery device is described in detail in U.S. 2011/0106006, which is incorporated by reference herein.

Drug delivery devices similar to that shown in FIGS. 4A-4B were used in a study, described in Example 4, to assess release of incretin mimetic from formulations (aqueous suspensions) of the incretin mimetic and an organic acid. The reservoir of devices were filled with either a control formulation (˜20 mg/mL exendin-4 in PBS) or with a formulation containing an aqueous suspension of exendin-4 (˜20 or 50 mg/mL) and an organic acid (e.g., p-aminobenzoic acid, p-coumaric acid, or terephthalic acid; 20 mg per device). The devices were submerged into polypropylene tubes containing phosphate buffered saline and incubated at 37° C. Approximately once per day, the devices were exchanged into fresh buffer, and device-exposed solutions were recovered for analysis.

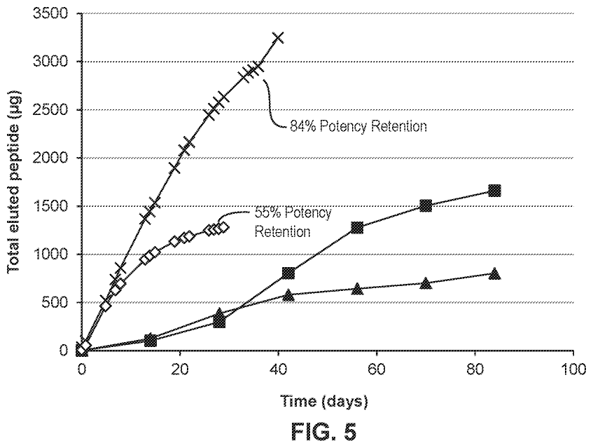

FIG. 5 shows the total quantity of eluted incretin mimetic (average cumulative release), in micrograms, as a function of number of elapsed days, from devices comprising aqueous suspensions of exendin-4 and p-aminobenzoic acid (closed squares), p-coumaric acid (triangles), or terephthalic acid (x symbols). Release of exendin-4 from control devices filled with exendin-4 in PBS (i.e., a control formulation lacking an organic acid) is indicated (diamonds).

After operating for a set period (˜30, 40, 60, or 90 days), selected devices were sacrificed for mass balance measurements. Fluid samples were extracted from the device reservoir and peptide content was quantified by HPLC. The quantity of retained peptide was added to the quantity of released peptide (calculated from weekly release data by trapezoidal summation) and compared to the initial mass of peptide loaded into the device reservoir. As indicated in FIG. 5, in devices comprising a composition that included an organic acid (terephthalic acid), approximately 84% of the initially loaded peptide was recovered in an intact form after 40 days. In contrast, in the devices containing a control formulation lacking an organic acid, approximately 55% of the initially loaded peptide was recovered in an intact form after 30 days. Devices comprising a composition with a stabilizing organic acid produced a sustained output of chemically intact, potent, active peptide relative to devices with a control formulation lacking the organic acid.

The data in FIG. 5 shows that compositions (and devices comprising the composition) comprising an organic acid with a water solubility at room temperature of less than about 10 g/mL and that maintains pH of the composition in its environment of use at between about 3.0-6.0 stabilize the incretin mimetic to provide release of the incretin mimetic in its chemically intact, biologically active form at a constant rate and for a longer period of time than do compositions without an organic acid. The stabilization of the incretin mimetic by the organic acid is supported by the mass balance measurements (released+retained incretin memetic) conducted in the data discussed with respect to FIG. 5, where devices with a composition comprising an organic acid and devices with a composition lacking an organic acid had eluted a small fraction (˜10-20%) of initially loaded peptide after 30-40 days. Approximately 84% of the initial peptide load could be recovered from devices containing an organic acid as a stabilizing agent after ˜40 days; only 55% of the initial peptide load could be recovered from control devices after ˜30 days. This difference primarily reflects a loss of incretin mimetic peptide potency within the composition in the device interior. Devices with a non-acidic control composition may experience early shut down as decomposition products become a larger fraction of the diffusing solute mass, or the concentration of active peptide within the reservoir becomes too low to effectively saturate the number of available membrane pores, or decomposition products foul the membrane surface.

In embodiments where the composition is within a reservoir of a drug delivery device, it will be appreciated that the device when placed in its environment of use is open to the environment of use. That is, the environment of use and the composition in the device are in fluid communication via the pore or membrane pores in the drug delivery device.

Other drug delivery devices than that depicted in FIGS. 4A-4B are known in the art. The compositions described herein are useful for a variety of devices, including those that comprise a drug reservoir for retaining the incretin mimetic and organic acid formulation and those that have a substrate or matrix that can hold or contain the formulation. Controlled drug release devices suitable for use in the present invention generally can provide for delivery of the drug from the device at a selected or otherwise patterned amount and/or rate to a selected site in the subject. The drug delivery device must be capable of containing an amount of the formulation to provide a therapeutically effective amount of the incretin mimetic for the period of delivery. The period of delivery will vary according to the incretin mimetic, the condition being treated, and the individual patient. In one embodiment, the period of delivery, also referred to herein as a sustained period of time, intends a period of at least about two weeks to about six months. In another embodiment, a sustained period of time intends a period of at least about two weeks, or at least about three weeks, or at least about four weeks to about six months, or to about four months, or to about three months. In another embodiment, a sustained period of time intends a period of at least about 15 days, or at least about 21 days, or at least about 30 days, or at least about 45 days, or at least about 60 days. In other embodiments, the period of time is from about 2 hours to about 72 hours, from about 4 hours to about 36 hours, from about 12 hours to about 24 hours, from about 2 days to about 30 days, from about 5 days to about 20 days, from about 7 days or more, from about 10 days or more, from about 100 days or more; from about 1 week to about 4 weeks, from about 1 month to about 24 months, from about 2 months to about 12 months, from about 3 months to about 9 months, from about 1 month or more, from about 2 months or more, or from about 6 months or more.

Accordingly, in another aspect, an implantable device is contemplated. The device comprises a reservoir comprising a formulation of an incretin mimetic, the formulation comprising (i) an amount of the incretin mimetic to provide substantially zero-order release of the incretin mimetic for a delivery period of at least about 30 days and at a rate that provides a therapeutic effect and (ii) an organic acid that (a) maintains a pH of the formulation when hydrated in its environment of use of between 3.0-6.0 for the delivery period and (b) is present at the end of the delivery period in an amount approximately equal to or above its saturation concentration in the formulation when hydrated.

In one embodiment, the formulation comprising an incretin mimetic and an organic acid is in a dry form. For example, the dry formulation may be present in the reservoir of a device as a powder, a tablet or a film. The device when in use, in vitro or in vivo, imbibes fluid from the surrounding environment to hydrate the dry formulation, thus forming in situ an aqueous suspension.

In one embodiment, the formulation when hydrated is characterized in that less than 30% of the incretin mimetic degrades when stored for 3 months at 37° C. It can be appreciated that the incretin mimetic should remain sufficiently stable over the period of delivery so that it retains sufficient potency to provide a therapeutically effective amount of incretin mimetic for the period of delivery.

The drug delivery device can be implanted at any suitable implantation site using methods and devices well known in the art. As noted infra, an implantation site is a site within the body of a subject at which a drug delivery device is introduced and positioned. Implantation sites include, but are not necessarily limited to a subdermal, subcutaneous, intramuscular, or other suitable site within a subject's body. Subcutaneous implantation sites are preferred because of convenience in implantation and removal of the drug delivery device. Exemplary subcutaneous delivery sites include external subcutaneous sites (e.g., under the skin of the arm, shoulder, neck, back, or leg) and internal subcutaneous sites within a body cavity. Methods for implanting or otherwise positioning drug delivery devices for subcutaneous delivery of a drug are well known in the art. In general, placement of the drug delivery device will be accomplished using methods and tools that are well known in the art, and performed under aseptic conditions with at least some local or general anesthesia administered to the subject.

### Methods of Treatment

In other aspects, methods of treatment using the compositions and devices described herein are contemplated. In one embodiment, a method for sustained, controlled delivery of an incretin mimetic is contemplated, where a composition or a delivery device comprising a composition as described herein is provided. Incretin mimetics are commercially approved for treatment of a variety of conditions, some of which are now described.

In one embodiment, a method to lower plasma glucose or to treat diabetes mellitus is contemplated, by providing or administrating a composition or a delivery device comprising a composition as described herein. Diabetes mellitus is a serious metabolic disease that is defined by the presence of chronically elevated levels of blood glucose (hyperglycemia). The term diabetes mellitus encompasses several different hyperglycemic states. These states include Type I (insulin-dependent diabetes mellitus or IDDM) and Type II (non-insulin dependent diabetes mellitus or NIDDM) diabetes. The hyperglycemia present in individuals with Type I diabetes is associated with deficient, reduced, or nonexistent levels of insulin which are insufficient to maintain blood glucose levels within the physiological range. Treatment of Type I diabetes involves administration of replacement doses of insulin, generally by a parenteral route. The hyperglycemia present in individuals with Type II diabetes is initially associated with normal or elevated levels of insulin; however, these individuals are unable to maintain metabolic homeostasis due to a state of insulin resistance in peripheral tissues and liver and, as the disease advances, due to a progressive deterioration of the pancreatic 13 cells which are responsible for the secretion of insulin. Thus, initial therapy of Type II diabetes may be based on diet and lifestyle changes augmented by therapy with an incretin mimetic.

Other uses for the compositions and devices herein include reducing food intake or reducing body weight, or a method for chronic weight management. The compositions and devices also find use in a method to reduce gastric motility or delay gastric emptying.

### III. Examples

The following examples are illustrative in nature and are in no way intended to be limiting.

### Example 1

### Compositions Comprising an Incretin Mimetic and an Organic Acid

The incretin mimetic exendin-4 was obtained commercially. Aqueous heterogeneous solutions of exendin-4 were prepared by combining 1 mL of a solution of the peptide (2 mg/mL in phosphate buffer saline, pH 7.4, combined concentration of phosphate species ˜5 mM) with approximately 20 mg of one of the following solid, partially water-soluble organic acids: p-coumaric acid, m-coumaric acid, p-methoxycinnamic acid, trans-cinnamic acid, 4-methylcinnamic acid, 4-aminobenzoic acid (PABA), or citric acid (water soluble acid, control). All formulations were prepared in triplicate. Aliquots of the stock solution of exendin-4 in phosphate buffered saline (3×1 mL) were retained as an additional control. All formulations (active and control) were incubated in polypropylene tubes at 37° C. At selected time points (approximately once per week), a small sample (˜40 μL) was withdrawn from each formulation, diluted with 360 μL of 1:1 acetonitrile/water+0.1% TFA, and analyzed by reverse-phase HPLC. The signal corresponding to exendin-4 was integrated at 220 nm and compared to a standard curve to quantify the peptide present. This data was plotted over time to track changes in both absolute and relative potency (i.e., versus t=0). Additionally, the relative potency data was plotted logarithmically and found to approximate a first-order decay process for 1-2 months following mixture assembly. Selected results are shown in FIG. 1, where data for the formulations is indicated as follows: p-coumaric acid (closed squares), m-coumaric acid (x symbols), p-methoxycinnamic acid (closed triangles), trans-cinnamic acid (open triangles), 4-methylcinnamic acid (asterisks), 4-aminobenzoic acid (PABA) (open circles), citric acid (water soluble acid, as a control, closed diamonds), PBS control (open diamonds). Formulations comprising organic acids with limited water solubility of less than 10 g/L at room temperature and that maintained a pH value of the suspension between about 4.0 and 5.4 enhance stability of the peptide relative to the suspensions lacking an organic acid.

### Example 2

### Comparison of Formulations Containing Different Concentrations of Exendin-4

Exendin-4 was sourced commercially. Four aqueous formulations were prepared to either include or exclude (control formulations) a solid organic acid, p-coumaric acid, at ˜20 mg/mL to greatly exceed its saturation point in phosphate-buffered saline, at two concentrations of exendin-4—approximately 2.0 mg/mL or 0.4 mg/mL. Each formulation was prepared in triplicate and incubated in sealed polypropylene tubes at 37° C. Stability of the peptide in each formulation was assessed by quantifying the peptide present in formulation aliquots via reverse-phase HPLC (as described in Example 1) at selected time points. Data from the first 30-40 days of the study is shown in FIG. 2, where the control (lacking an organic acid) formulations are denoted by circles, with open circles corresponding to the formulation with an exendin-4 concentration of 2 mg/mL and closed circles to the formulation with exendin-4 concentration of 0.4 mg/mL, and the organic-acid containing formulations are denoted by squares, with open squares corresponding to the formulation with an exendin-4 concentration of 2 mg/mL and closed squares to the formulation with exendin-4 concentration of 0.4 mg/mL. The Y-axis in FIG. 2 shows the log of the potency retention ratio of the peptide (defined as the quantity of intact peptide remaining at each time point divided by the initial peptide load at time zero).

### Example 3

### Preparation of Additional Formulations Containing Exendin-4 and Organic Acids

Exendin-4 was sourced commercially. A number of additional exendin-4 formulations were constructed in triplicate as described in Example 1 to include the acids listed in Table 1. Formulations were similarly stored in polypropylene tubes at 37° C., and aliquots were similarly analyzed by HPLC at selected time points to measure the rate of peptide decomposition over a window of ≥30 days. Apparent first order rate constants were calculated for each formulation and plotted against the corresponding formulation pH, as shown in FIG. 3. Data from formulations that retain >70% of the initial peptide potency after 30 days are marked with diamonds; data from formulations that retain <70% of the initial peptide potency after 30 days are marked with squares. This study suggests a strong relationship between peptide stability and pH. Formulations with pH values between 4 and 5 were generally much more chemically stable than the control formulation (PBS, pH=7.2-7.4) or to formulations containing a higher concentration of acid.

### Example 4

### Drug Delivery Device Comprising a Composition with an Incretin Mimetic and an Organic Acid

Drug delivery devices like those shown in FIG. 4A were assembled in triplicate to contain either a control formulation (˜20 mg/mL exendin-4 in PBS) or a formulation containing a solution of exendin-4 (˜20 or 50 mg/mL, depending on the device set) combined with a selected stabilizing acid (e.g., p-aminobenzoic acid, p-coumaric acid, or terephthalic acid; 20 mg per device). Mesoporous alumina membranes were employed in order to constrain the diffusion rate of the peptide. Following assembly and loading, devices were submerged into polypropylene tubes containing phosphate buffered saline and incubated at 37° C. Approximately once per day, the devices were exchanged into fresh buffer, and device-exposed solutions were sealed and archived at −80° C. until analysis. Analysis was conducted upon selected time points using RP-HPLC, LC-MS, and/or ELISA as appropriate, with peptide concentrations determined by means of appropriate standard curves. Selected results are shown in FIG. 5, where the x-axis depicts the number of elapsed days corresponding to each time point; the y-axis depicts the total quantity of eluted peptide (average cumulative release) within each sample set as expressed in micrograms; and the data points correspond as follows: devices with control formulation (diamonds); devices with terephthalic acid formulation (x symbols) p-aminobenzoic acid formulation (squares); devices with p-coumaric acid formulation (triangles).

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope.

1. A composition, comprising:
an aqueous suspension comprising
an incretin mimetic, and
an organic acid that (i) has a water solubility at room temperature of between about and 10 g/L, (ii) a molar mass of less than about 500 grams per mole, and (iii) maintains a pH of the suspension in its environment of use of between 3.0-6.0 for a period of at least about 30 days.

2. The composition of claim 1, wherein the organic acid is present in an amount equal to or above its saturation concentration at the end of the period.

3. The composition of claim 1, wherein the incretin mimetic is a glucagon-like peptide-1 (GLP-1) agonist.

4. The composition of claim 1, wherein in the GLP-1 agonist is exendin or an exendin-analogue.

5. The composition of claim 4, wherein the GLP-1 agonist is exendin-4.

6. The composition of any claim 1, wherein the incretin mimetic at the beginning of the period is at a concentration in the solution of greater than or equal to 1 mg/mL.

7. The composition of claim 1, wherein the organic acid is an aromatic carboxylic acid.

8-9. (canceled)

10. The composition of claim 7, wherein the carboxylic acid is one having a benzene ring and one electron-donating group with antioxidant properties.

11. The composition of claim 10, wherein the carboxylic acid is selected from the group consisting of o-anisic acid, m-anisic acid, p-anisic acid; p-aminobenzoic acid (PABA), o-aminobenzoic acid (anthranilic acid), o-toluic acid, m-toluic acid, p-toluic acid and salicylic acid.

12. The composition of claim 7, wherein the carboxylic acid is one having one or two carboxylic acid groups directly bonded to a biphenyl ring system.

13. The composition of claim 12, wherein the carboxylic acid is selected from the group consisting of 2-phenylbenzoic acid, 3-phenylbenzoic acid, 4-phenylbenzoic acid and diphenic acid.

14-21. (canceled)

22. The composition of claim 7, wherein the carboxylic acid is a cis-cinnamic acid or a trans-cinnamic acid.

23. The composition of claim 22, wherein the carboxylic acid is a trans-cinnamic acid with one or two electron-donating groups selected from hydroxy, methoxy, amino, alkylamino, dialkylamino, or alkyl groups.

24. The composition of claim 23, wherein the trans-cinnamic acid is selected from the group consisting of o-coumaric acid, m-coumaric acid, p-coumaric acid, o-methylcinnamic acid, m-methylcinnamic acid, p-methylcinnamic acid; o-methoxycinnamic acid, m-methoxycinnamic acid, and p-methoxycinnamic acid; and ferulic acid.

25-28. (canceled)

29. The composition of claim 1, wherein the organic acid is a carboxylic acid containing an aromatic ring.

30. The composition of claim 29, wherein the aromatic carboxylic acid is selected from the group consisting of 3-phenylpropionic acid, cinnamic acid, a hydroxy-derivative of cinnamic acid, a methoxy derivative of cinnamic acid, nicotinic acid, benzoic acid, an amino-derivative of benzoic acid, a methoxy derivative of benzoic acid, and terephthalic acid.

31. The composition of claim 30, wherein the hydroxy-derivative of cinnamic acid is m-coumaric acid or p-coumaric acid.

32. The composition of claim 30, wherein the amino-derivative of benzoic acid is 2-aminobenzoic acid (anthranilic acid) or 4-aminobenzoic acid (para-aminobenzoic acid; PABA).

33. A device, comprising: a composition according to claim 1, wherein the device is configured for subcutaneous implantation into a mammal.

34. A method to lower plasma glucose or to treat diabetes mellitus, comprising:
providing a composition according to claim 1.
